(19) United States
(12) Patent Application Publication
(10) Pub. No.: US 2008/0014600 A1
(43) Pub. Date: Jan. 17, 2008

(54) DIAGNOSIS OF MULTIPLE SCLEROSIS AND OTHER DEMYELINATING DISEASES

(75) Inventor: Aristo VOJDANI, Los Angeles, CA (US)

Correspondence Address: KNOBBE MARTENS OLSON & BEAR LLP, 2040 MAIN STREET, FOURTEENTH FLOOR, IRVINE, CA, 92614, US

(73) Assignee: IMMUNOSCIENCES LAB., INC., Beverly Hills, CA (US)

(21) Appl. No.: 11/780,447

(22) Filed: Jul. 19, 2007

Related U.S. Application Data
(62) Division of application No. 10/233,892, filed on Aug. 29, 2002, now abandoned.

(51) Int. Cl. G01N 33/00

(52) U.S. Cl. 435/7.92

(57) ABSTRACT

A method of diagnosing multiple sclerosis and other demyelinating diseases or predicting a predisposition to multiple sclerosis and other demyelinating diseases. The method utilizes detection of increased amounts of memory lymphocytes reacting to MS antigens, proinflammatory cytokines, and antibodies against MS antigens.

### RELATED APPLICATION

This application is a divisional of U.S. patent application Ser. No. 10/233,892, filed Aug. 29, 2002, the entire contents of which are incorporated herein by reference.

### REFERENCE TO SEQUENCE LISTING

The Sequence Listing in the present application is identical to the Sequence Listing currently on file in the parent application (Ser. No. 10/233,892, filed Aug. 29, 2002) and is incorporated herein by reference in its entirety. In accordance with 37 CFR 1.821(e), please use the last filed CRF Sequence Listing filed in that application as the CRF Sequence Listing for the instant application. It is understood that the Patent and Trademark Office will make the necessary change in application number and filing date for the instant application.

### BACKGROUND OF THE INVENTION

1. Field of the Invention

The invention relates to a method of diagnosing multiple sclerosis and other demyelinating diseases.

2. Description of the Related Art

Autoimmune neurologic disorders occur when immunologic tolerance to myelin and other neurologic antigens of the Schwann cell, the axon and the motor or ganglioside neuron are lost. The resulting demyelinating diseases share the pathologic features of destruction of myelin, accompanied by an inflammatory infiltration in the brain, spinal cord, or the optic nerve. Based on the location of the lesions, the occurrence of relapses, and the nature of events, it is possible to separate the clinical neurologic syndromes of multiple sclerosis, acute disseminated encephalomyelitis, acute transverse myelitis, and optic neuritis (1,2).

The most common demyelinating disease is multiple sclerosis. Multiple sclerosis (MS) is a disease of the myelin central nervous system (CNS) that is clinically characterized by episodes of neurologic dysfunction separated by time and space.

Currently, there is no specific diagnostic test for Multiple Sclerosis (“MS”). The diagnosis is made based on clinical grounds, which may vary from clinician to clinician. Supportive evidence to clinical grounds can come from the MRI of the brain, cerebrospinal fluid studies, and evoked response (1,5).

MRI is usually the procedure of choice for corroboration of a clinical diagnosis of MS, particularly when gadolinium enhancement is used. High signal intensity lesions on T-2 weighted images, particularly in the periventricular areas, support a diagnosis of MS. MRI is not specific for MS, since several diseases of the white matter such as ischemic, infectious, metabolic and neoplastic present similar pictures.

Cerebrospinal fluid examination is an additional supportive technique for the diagnosis of MS. CSF total protein is usually normal but CSF IgG levels may be increased and the ratio of CSF IgG to CSF albumin is often elevated. The presence of discrete IgG oligoclonal band by immunofixation electrophoreses is more characteristic but not specific for MS. This oligoclonal band may be found in many conditions including: subacute sclerosing panencephalitis, neurosyphilis, Lyme Disease, HTLV-1 associated myelopathy, Sjögren Syndrome, sarcoidosis, meningeal carcinomatosis and HIV infection.

The third technique for support in the diagnosis of MS is evoked response, which includes: pattern-sensitive visual-evoked potential, the brainstem auditory-evoked potential (5).

Overall, the combination of MRI, the CSF examination and evoked responses support a clinical diagnosis of MS in a majority of cases. However, all three determinants (MRI, CSF examination and evoked response) are not always positive in the same patient. For example, abnormal MRI alone or abnormal MRI with normal CSF and abnormal evoked response can challenge many clinicians over the diagnosis of MS. Hence, there is no definitive test available to diagnose multiple sclerosis.

Therefore, there is a need for additional markers to aid in the diagnosis of MS. These biomarkers become very useful when the immunological mechanisms behind the development of neurological dysfunction associated with MS are understood.

### SUMMARY OF THE INVENTION

The preferred embodiment provides a method for diagnosing the likelihood and severity of a demyelinating disease in a patient, comprising the steps of: a) determining a level of antibodies against a neuron-specific antigen in a sample from the patient; b) comparing the level of antibodies determined in step a) with a normal level of the antibodies, wherein (i) normal level of antibodies for neuron-specific antigen indicate optimal conditions; (ii) lower than normal level of antibodies for neuron-specific antigen indicate absence of the demyelinating disease; and (iii) higher than normal level of antibodies for neuron-specific antigen indicate a likelihood of the demyelinating disease.

Another preferred embodiment provides a method for diagnosing the likelihood and severity of a demyelinating disease in a patient, comprising the steps of: a) isolating peripheral blood mononuclear cells (PBMCs) from the patient; b) incubating PBMCs with a neuronal antigen or peptide; c) measuring a concentration of cytokines resulting from step b); and d) comparing the concentration of cytokines determined from step c) with a normal level of cytokines, wherein (i) normal level of cytokines for the neuronal antigen or peptide indicate optimal conditions; (ii) lower than normal level of cytokines for the neuronal antigen or peptide indicate absence of the demyelinating disease; and (iii) higher than normal level of cytokines after challenge with the neuronal antigen or peptide indicate a likelihood of the demyelinating disease.

Another preferred embodiment provides a method for diagnosing the likelihood and severity of a demyelinating disease in a patient, comprising the steps of: a) isolating peripheral blood mononuclear cells (PBMCs) from the patient; b) incubating PBMCs with neuronal antigen or peptide; c) determining an amount of neuronal antigen- or peptide-specific activated T-cells or neuronal-specific memory lymphocytes resulting from step b); d) obtaining a stimulation index from step c); and e) comparing the stimulation index from step d) with a normal stimulation index, wherein (i) normal stimulation index indicates optimal conditions; (ii) lower than normal stimulation index indicates absence of the demyelinating disease; and (iii) higher than normal stimulation index indicates a likelihood of a demyelinating disease.

The present invention also provides a method for diagnosing the likelihood and severity of multiple sclerosis in a patient, comprising the steps of: a) determining a level of antibodies against α-β-crystallin in a sample from the patient; b) comparing the level of antibodies determined in step a) with a normal level of the antibodies in control patients; c) determining an amount of neuronal α-β-crystallin-specific activated T-cells or neuronal-specific memory lymphocytes in a sample from the patient; d) obtaining a stimulation index from step c); and e) comparing the stimulation index from step d) with a normal stimulation index, wherein: (i) a normal level or lower than normal level of both stimulation index and antibodies for the α-β-crystallin indicate control patient conditions; (ii) higher than normal levels of either stimulation index or antibodies for the α-β-crystallin indicate a possibility of multiple sclerosis; and (iii) higher than normal levels of both stimulation index and antibodies for the α-β-crystallin indicate a likelihood of multiple sclerosis. In one embodiment, the normal level of antibodies is calculated by taking a mean of levels of antibodies in individuals without symptoms relating to multiple sclerosis. In another embodiment, the higher than normal level of antibodies is higher than about two standard deviations of normal levels of antibodies in a control group. In one embodiment, the determining the level of antibodies in any or all of steps a) and b) is accomplished using an immunoassay. In another embodiment, the immunoassay is an enzyme-linked immunosorbent assay. The antibodies may be IgG, IgA or IgM. In one embodiment, the normal stimulation index is calculated by taking a mean of stimulation indices in individuals without symptoms relating to multiple sclerosis. In one embodiment, the higher than normal stimulation index is higher than about two standard deviations of a normal stimulation index of a control group. In another embodiment, the T-cells are antigen-specific CD3 activated T-cells. The method may further comprise the step of obtaining a clinical test result including MRI, evoked response or cerebrospinal fluid. In one embodiment, the method further comprises a step of obtaining at least two clinical test results from MRI, evoked response and cerebrospinal fluid. In another embodiment, the method further comprises a step of obtaining clinical test results of MRI, evoked response and cerebrospinal fluid. In another embodiment, the diagnosis is made according to Table 2.

### BRIEF DESCRIPTION OF THE DRAWINGS

FIG. 1 is a diagram that shows the regulation of Th1/Th2 responses by the balance or imbalance between microglia and astrocytes in demyelinating processes. The right panel demonstrates negative regulation of astrocytes by induction of T-helper-2 cell responses and production of growth factors. The left panel represents positive regulation of microglia by induction of T-helper-1 cell responses and production of proteases TNF-α and nitric oxide, which results in demyelination of neurons. The astrocytic factors, such as TGF-β and TGE₂, through a negative feedback mechanism on microglia, prevent the propagation of Th1 responses, while interferon-γ secreted by activated microglia and Th1 cells induces the production of TGF-β and PGE₂, which in turn results in downregulation of Th1 cell responses and the prevention of demyelination.

FIG. 2 is a diagram that shows apoptosis of activated T-cells by means of immunoregulatory mechanism, which prevents tissue damage.

FIG. 3 is a diagram that shows cellular and humoral immune mechanisms in stress, infection and toxic chemical-induced neurotoxicity, which includes neuronal degeneration, secondary demyelination, and reactive astrogliosis.

FIG. 4 is a diagram that shows a procedure for detection of myelin and other antigen-specific CD4 T-cells in patients with possible neuroimmunologic disorders.

FIG. 5 is a graph that shows percent elevation in IgG, IgM, and IgA antibodies against three different neurological antigens in controls and patients with multiple sclerosis at cut-off values above about 2 standard deviations of the mean of the controls.

FIG. 6 is a graph showing an in vitro stimulation study of myelin-specific lymphocytes in controls and patients with possible multiple sclerosis.

FIG. 7 is a graph showing measurement of Th-1, Th-2, and proinflammatory cytokine production in blood samples of two different controls and two patients with possible multiple sclerosis.

FIG. 8 is a graph showing percent elevation of different cytokines produced by MBP-reactive T-cells in controls and patients with multiple sclerosis at cut-off values above 2 standard deviations of the mean of the controls.

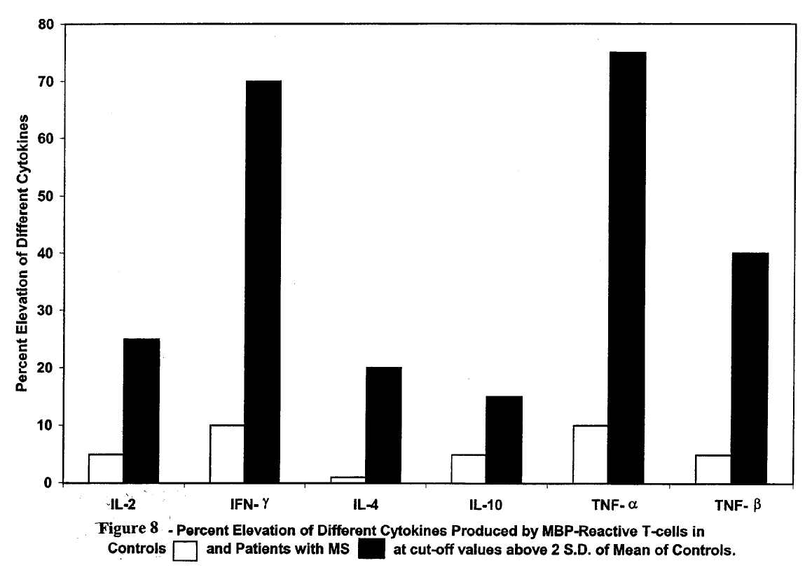

### DETAILED DESCRIPTION OF THE PREFERRED EMBODIMENT

The hallmark of the MS lesion is a plaque, an area of demyelination sharply demarcated from the usual white matter shown in MRI scans. The histological appearance of the plaques varies in different stages of the disease. In active lesions, the blood-brain barrier is damaged, thereby permitting extravasation of serum proteins into the extracellular space. Inflammatory cells can be seen in perivascular cuffs and throughout the white matter. Activated monocyte-derived macrophages and activated lymphocytes predominate. CD4 T-cells, especially T-helper-1 (but not CD8 cells) accumulate around postcapillary venules at the edge of the plaque and are also scattered in the white matter (3-5). In active lesions, up-regulation of adhesion molecules and markers of lymphocyte and monocyte activation, such as IL2-R and CD26 have also been observed. Demyelination in active lesions is not accompanied by destruction of oligodendrocytes. In contrast, in the chronic phase of the disease, the lesions are characterized by the loss of oligodendrocytes and hence, the presence of myelin oligodendrocytes glycoprotein (MOG) antibodies in the blood. T-cells bearing the γ-δ T-cell receptor are found in MS lesions and may be involved in the selective destruction of oligodendrocytes. The γ-δ T-cells are reacting with heat shock proteins (HSP65), such as α-β crystalline which may be found in oligodendrocytes under stressful conditions. This particular reaction of γ-δ T-cells with oligodendrocytes results in selective cellular destruction, the release of α-β crystallin into circulation, the presentation of macrophages and T-cells, and the production of specific antibodies against myelin oligodendrocyte glycoprotein (MOG) and α-β crystallin (6-14).

The activated helper T-cells that are CD45RA (phenotype associated with memory or activated T-cells) accumulate in the brain and spinal cord of MS sufferers. These findings imply that activated T-cells, activated monocytes/macrophages and their cytokines have a special role in the pathogenesis of the disease (15-20). Activated T-helper cells release interleukin-2, interferon-γ and lymphotoxins, while monocytes release tumor necrosis factor-α (TNF-α). The monocytes are primed by T-cell-derived interferon-γ to release TNF-α. TNF-α and lymphotoxins have been reported to be injurious to myelin and oligodendrocytes. Indeed, it can be said that lymphotoxins or TNF-β can cause apoptosis of cultured oligodendrocytes (20-26). Thus, the liberation of toxic cytokines by monocytes and T-helper-1 cells, coupled with macrophage activation with release of free radicals, may ultimately culminate in the destruction of myelin in MS.

The Role of Th1/Th2 Cytokines, Microglia and Astrocytes in Regulating Immune Responses and the Development of Neuropathologies

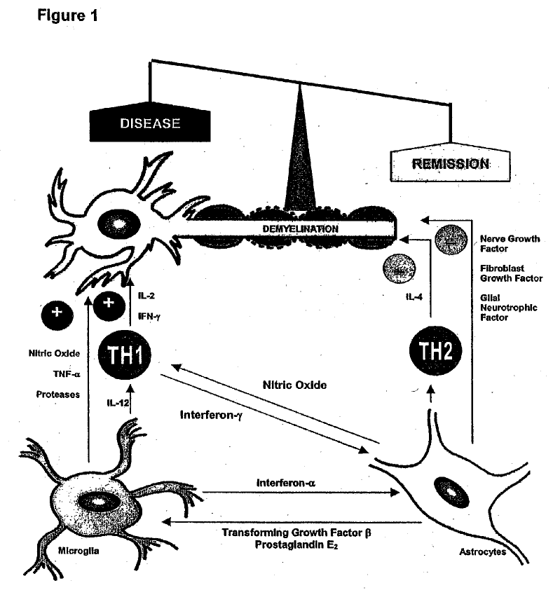

T-helper-1 (Th1) and Th2 cells can be redefined as polarized forms of immune responses that not only represent a useful model for understanding the pathogenesis of several diseases, but also one that can provide the basis for the development of immunotherapeutic strategies. Mechanisms that regulate the balance of Th1 and Th2 cells, such as cytokines, are of great interest because they can determine the outcome of the disease. For example, interleukin-12 (IL-12) promotes the development of Th1 cells, whereas IL-4 leads to the expansion of Th2 cells. In CNS inflammation, it has been shown that there might be a balance between microglia and astrocytes in regulating local immune reactions, including Th1/Th2 responses (21-24). This positive and negative regulation of Th1/Th2 by the microglia and astrocytes is shown in FIG. 1.

As shown in the FIG. 1, microglia produces IL-12, which primarily promotes the development of Th1 cells. Astrocytes cannot produce IL-12 and induce mainly Th2-cell responses, thereby representing important homeostatic mechanisms during recovery from Th1-mediated inflammation (21, 22,27-30).

The capacity of microglia and astrocytes to stimulate Th1 and Th2 cells depends on their surface molecules, such as MHC class II, B7 and CD40. MHC class II-positive microglia directly induce encephalitogenic myelin basic protein (MBP)-reactive CD4⁺ T-cells to produce interferon-γ (IFN-γ) and TNF-α in vivo. After treatment with IFN-γ and/or bacterial antigens (LPS), microglia express CD40, which contributes to Th1 activation (31-33).

Th1 cells can stimulate microglia to produce prostaglandin E₂ (PGE₂), which provides a negative feedback mechanism for downregulation of Th1-cell responses within the CNS. During antigen presentation within the CNS, IFN-γ secreted by activated microglia and Th1 cells can induce astrocytes to secrete PGE₂ and contribute to the downregulation of microglia and Th1-cell responses (34,35). Lymphocyte reaction to myelin and other neurologic antigens

The major question, then, is “What triggers the influx of activated T-cells and monocytes into the CNS?” Considerations include a failure of immunoregulation between astrocytes and microglia that permits T-cells specific for myelin antigens to be induced and to enter the CNS (13). One way of examining this question is to study an experimental animal model that resembles the human disease MS. EAE, an animal disease induced by immunization with spinal cord homogenate or myelin proteins or by the adoptive transfer of T-cells reactive to myelin antigens, shares many features with MS. The disease declares itself as an ascending paralysis, characterized by weakness of the tail, which is followed by paralysis of the hind limbs and the fore limbs (19-21). This adoptive transfer of EAE to healthy animals with sensitized lymphocytes from sick animals clearly indicates that neurologic, antigen-specific T-lymphocytes can actually induce disease. In fact, many investigations have shown that if myelin-specific CD4 Th1 type (which produces IL-2, IFN-γ, LT and TNF-α) is adoptively transferred to the naive animal, EAE will be induced. Thus, the myelin antigen-specific CD4 T-cells are central to the initiation of demyelinating diseases (19,24,26).

Kinetic studies have shown that after the transfer of CD4, Th1 cells reactive to MBP are the first cells to infiltrate the central nervous system and are detected within four to five days after the transfer. As the lesion evolves, the MBP-specific CD4 Th1 cells constitute only between 1%-3% of the infiltrating cells, thereby indicating recruitment of other mononuclear cells. Activated lymphocyte to other myelin components, such as proteolipid protein (PLP), is equally important in the pathogenesis of demyelinating diseases (15-20).

In addition to Th1, Th2 and proinflammatory cytokines abnormalities and myelin antigen-specific CD4 T-cell evaluation, a number of other immune regulation abnormalities have been reported to occur in the blood and spinal fluid of MS patients. An increase in IgG and the occurrence of oligoclonal bands representing restricted populations of antibodies in the spinal fluid is a consistent finding. While the antigens with which the oligoclonal band antibodies react are not known, recent evidence has clearly identified antigens such as myelin basic protein, myelin oligodendrocyte glycoprotein and α-β crystallin against which the autoimmune response in MS is directed.

With immunogold-labeled peptides of myelin antigens and high-resolution microscopy, techniques that can detect antigen-specific antibodies in situ, scientists have identified autoantibodies specific for the central nervous system myelin antigen myelin/oligodendrocyte glycoprotein. These autoantibodies were specifically bound to disintegrating myelin around axons in lesions of acute multiple sclerosis and the marmoset model of allergic encephalomyelitis. These findings represent direct evidence that autoantibodies against a specific myelin protein mediate target membrane damage in central nervous system demyelinating disease (18-20).

In the complete collection of proteins extracted from MS-affected myelin, the dominant human antigen for CD4⁺ T-cells appears to be α-β crystalline a small heat shock protein. Enhanced levels of α-β crystallin are present in the cytosol of oligodendrocytes and astrocytes in MS lesions, where it is up-regulated at the earliest stages of lesional formation. After myelin phagocytosis in MS lesions, α-β crystallin becomes available to T-cells, suggesting the important role of this autoantigen in the pathogenesis of MS. The presentation of these antigens by T-cells to B-cells results in autoantibody production. It can therefore be said that IgG, IgM and IgA antibodies against myelin basic protein, myelin associated glycoprotein, myelin oligodendrocyte glycoprotein, proteolipid protein, phosphodiesterase, transaldolase, glutamate receptor, S-100 protein, small heat shock protein, such as α-β-crystallin, and other antigens, can aid in the diagnosis of MS and other demyelinating diseases.

Immunological Mechanisms of Injury in Multiple Sclerosis

Based on a review of the literature and results presented here, we propose that the following chain of events may lead to MS.

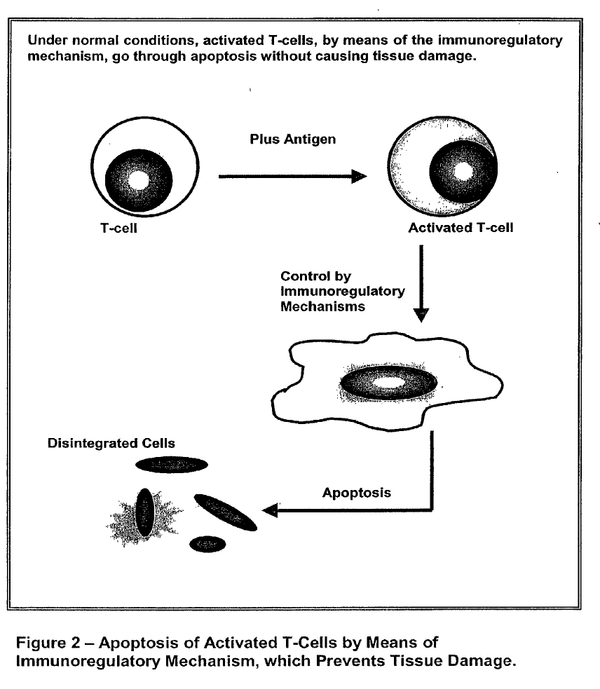

As a result of molecular mimicry and sequence homology between autoantigens and bacterial, viral or parasitic antigens, autoantibodies and autoreactive T-cells are generated in the blood. Under normal conditions, these autoreactive T-cells go through programmed cell death without causing any tissue damage, as shown in FIG. 2. However, for cross-reactive circulating T-cells and antibodies to become pathogenic, they can cross the blood-brain barrier.

Environmental factors such as stress, infections and toxic chemicals or their metabolites can disrupt the blood-brain barrier.

Viral particles, bacterial toxins, superantigens and reactive metabolites facilitate the movement and entrance of autoreactive T-cells and cross-reactive antibodies from the systemic circulation into the central nervous system.

In the central nervous system, the infectious agents antigens and toxic reactive metabolites up-regulate the expression of endothelial adhesion molecules, which further facilitates the entry of T-cells into the central nervous system.

Proteases, such as matrix metalloproteinases and others may further enhance the migration of autoreactive immune cells into the central nervous system by degrading extracellular-matrix macromolecules.

Through communication with macrophages, activated T-cells release significant amounts of proinflammatory cytokines, such as interferon-γ, tumor necrosis factor alpha and tumor necrosis factor beta.

Proinflammatory cytokines may directly damage the myelin sheath or up-regulate the expression of cell-surface molecules on neighboring lymphocytes and antigen-presenting cells.

Putative MS antigens, myelin basic protein, myelin proteolipid protein, myelin oligodendrocyte glycoprotein, myelin associated glycoprotein, α-β-crystallin phosphodiesterases and S-100 protein and other antigens are presented by macrophages with the help of MHC Class II, T-cell receptor and costimulatory molecules CD28-CTLA-4 to T-helper cells, which trigger enhanced immune response against one or all of MS antigens.

If this antigen presentation results in activation of T-helper cells and the production of proinflammatory cytokines, such as interferon-γ and TNF-α, it can trigger a cascade of events resulting in a proliferation of proinflammatory CD4 and T-helper-1 cells and ultimately cause further damage or injury to the myelin and oligodendrocytes.

Injury to the myelin and oligodendrocytes results in the proliferation of a significant amount of antigens into the circulation, which begins a vicious cycle of antibody (IgG, IgM, IgA) production against the MS antigens.

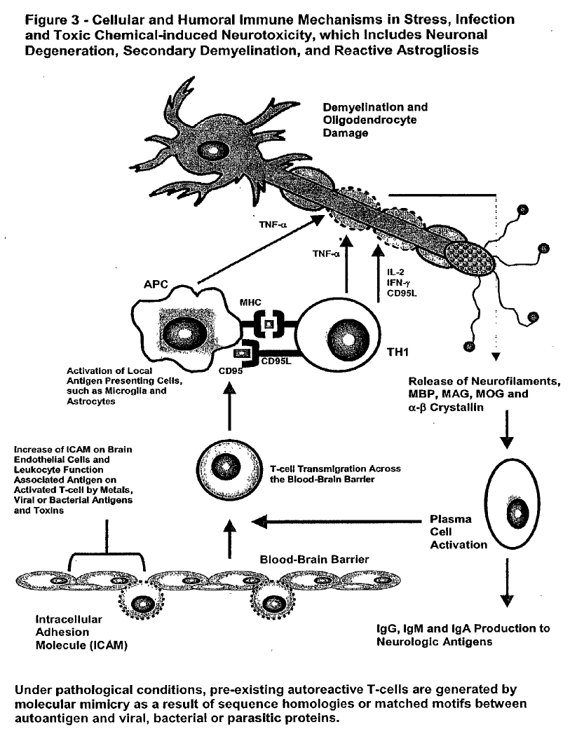

The binding of neuron-specific antibodies to myelin and oligodendrocytes and the formation of antigen-antibody complex with the involvement of complement cascades will induce antibody-dependent, cell-mediated cytotoxicity, apoptosis or death of neurons, which are observed as white spots in the MRI of the brain. A summary of these cellular and humoral immune mechanisms resulting in tissue damage is shown in FIG. 3.

This injury to the myelin membrane or the neurons results in axons that are no longer able to transit action potentials efficiently within the central nervous system. Blocking of the action potential results in the production of neurologic symptoms, which are detected by evoked responses (5).

Based on these immunological mechanisms, behind the injury to the neurons, it is possible to culture lymphocytes from patients with questionable MS and neurological antigens, and replicate a majority of these steps in a tissue culture environment. Only lymphocytes of MS patients, which possess prior memory of exposure to MS antigens in vivo, will be stimulated when they are exposed to MS antigens in the test tube. This will result in the production of a significant amount of proinflammatory cytokines, such as interferon-γ, TNF-α, TNF-β or all three cytokines.

Due to repeated injury to the neurons by cytokines, activated helper cells, macrophages, complement and proteases, neuron-specific antigens are released in the circulation. The release of these brain antigens and an initiation of immune response against them results in (IgG, IgM, IgA) antibodies in the blood of MS patients against one or all of the following MS antigens: myelin basic protein, myelin associated glycoprotein, myelin oligodendrocyte glycoprotein, proteolipid protein, phosphodiesterase, gangliosides, transaldolase, glutamate receptor, S-100 protein, glial fibrillary acidic protein, and small heat shock protein, such as α-β-crystallin.

The detection of a high percentage of lymphocytes reacting to MS antigen(s) and the production of a significant amount of proinflammatory cytokines in culture along with high levels of IgG, IgM or IgA antibodies against the neurologic antigen(s) will significantly enhance the sensitivity of MS detection.

The inventor has developed a laboratory test for diagnosing multiple sclerosis and other demyelinating diseases or predicting a predisposition to multiple sclerosis and other demyelinating diseases. The test utilizes detection of increased amounts of memory lymphocytes reacting to MS antigens, proinflammatory cytokines, and antibodies against MS antigens.

The preferred embodiment provides a method for diagnosing the likelihood and severity of a demyelinating disease in a patient, comprising the steps of: a) determining a level of antibodies against a neuron-specific antigen in a sample from the patient; b) comparing the level of antibodies determined in step a) with a normal level of the antibodies, wherein (i) normal level of antibodies for neuron-specific antigen indicate optimal conditions; (ii) lower than normal level of antibodies for neuron-specific antigen indicate absence of the demyelinating disease; and (iii) higher than normal level of antibodies for neuron-specific antigen indicate a likelihood of the demyelinating disease.

Another preferred embodiment provides a method for diagnosing the likelihood and severity of a demyelinating disease in a patient, comprising the steps of: a) isolating peripheral blood mononuclear cells (PBMCs) from the patient; b) incubating PBMCs with a neuronal antigen or peptide; c) measuring a concentration of cytokines resulting from step b); and d) comparing the concentration of cytokines determined from step c) with a normal level of cytokines, wherein (i) normal level of cytokines for the neuronal antigen or peptide indicate optimal conditions; (ii) lower than normal level of cytokines for the neuronal antigen or peptide indicate absence of the demyelinating disease; and (iii) higher than normal level of cytokines for the neuronal antigen or peptide indicate a likelihood of the demyelinating disease.

Another preferred embodiment provides a method for diagnosing the likelihood and severity of a demyelinating disease in a patient, comprising the steps of: a) isolating peripheral blood mononuclear cells (PBMCs) from the patient; b) incubating PBMCs with neuronal antigen or peptide; c) determining an amount of neuronal antigen- or peptide-specific activated T-cells or neuronal-specific memory lymphocytes resulting from step b); d) obtaining a stimulation index from step c); and e) comparing the stimulation index from step d) with a normal stimulation index, wherein (i) normal stimulation index indicates optimal conditions; (ii) lower than normal stimulation index indicates absence of the demyelinating disease; and (iii) higher than normal stimulation index indicates a likelihood of a demyelinating disease.

The laboratory tests are summarized in the following parts A-C, shown in Table 1.

TABLE 1
Part A: Test for memory lymphocytes reacting to MS antigens

| | |
|---|---|
| 1. | Myelin lymphocyte immune function assay to myelin basic protein (MBP) |
| 2. | Myelin lymphocyte immune function assay to myelin basic protein peptides |
| 3. | Myelin lymphocyte immune function assay to myelin oligodendrocyte glycoprotein (MOG) |
| 4. | Myelin lymphocyte immune function assay to myelin oligodendrocyte glycoprotein |
| | peptides |
| 5. | Myelin lymphocyte immune function assay to myelin associated glycoprotein (MAG) |
| 6. | Myelin lymphocyte immune function assay to myelin associated glycoprotein peptides |
| 7. | Myelin lymphocyte immune function assay to proteolipid protein (PLP) |
| 8. | Myelin lymphocyte immune function assay to proteolipid protein peptides |
| 9. | Myelin lymphocyte immune function assay to small heat shock protein small heat shock |
| | protein, such as α-β-crystallin |
| 10. | Myelin lymphocyte immune function assay to transaldolase |
| 11. | Myelin lymphocyte immune function assay to transaldolase peptides |
| 12. | Myelin lymphocyte immune function assay to glial fibrillary acidic proteins (GFAP) |
| 13. | Myelin lymphocyte immune function assay to S-100 proteins |
| 14. | Myelin lymphocyte immune function assay to cross-reactive peptides from dietary |
| | proteins and infectious agents |
| 15. | Myelin lymphocyte immune function assay to glutamate receptor |
| 16. | Myelin lymphocyte immune function assay to phosphodiesterase |

Part B: Test for proinflammatory cytokines

| | |
|---|---|
| 1. | Production of interleukin-2 or T-helper-1 cytokine |
| 2. | Production of interferon-γ or T-helper-1 cytokine after stimulation of lymphocytes with |
| | neuron-specific antigens |
| 3. | Production of tumor necrosis factor alpha or proinflammatory cytokines after stimulation |
| | of lymphocytes with neuron-specific antigens |
| 4. | Production of tumor necrosis factor beta or lymphotoxin (proinflammatory cytokine) after |
| | stimulation of lymphocytes with neuron-specific antigens |
| 5. | Production of interleukin-12 |

Part C: Test for antibodies against MS antigens

| | |
|---|---|
| 1. | Elevation of IgG, IgM, or IgA antibodies against myelin basic protein (MBP) |
| 2. | Elevation of IgG, IgM, or IgA antibodies against myelin basic protein peptides |
| 3. | Elevation of IgG, IgM, or IgA antibodies against myelin oligodendrocyte glycoprotein |
| | (MOG) |
| 4. | Elevation of IgG, IgM, or IgA antibodies against myelin oligodendrocyte glycoprotein |
| | peptides |
| 5. | Elevation of IgG, IgM, or IgA antibodies against myelin associated glycoprotein (MAG) |
| 6. | Elevation of IgG, IgM, or IgA antibodies against myelin associated glycoprotein peptides |
| 7. | Elevation of IgG, IgM, or IgA antibodies against proteolipid protein (PLP) |
| 8. | Elevation of IgG, IgM, or IgA antibodies against proteolipid protein peptides |
| 9. | Elevation of IgG, IgM, or IgA antibodies against small heat shock protein small heat shock |
| | protein, such as α-β-crystallin |
| 10. | Elevation of IgG, IgM, or IgA antibodies against transaldolase |
| 11. | Elevation of IgG, IgM, or IgA antibodies against transaldolase peptides |
| 12. | Elevation of IgG, IgM, or IgA antibodies against glial fibrillary acidic proteins (GFAP) |
| 13. | Elevation of IgG, IgM, or IgA antibodies against S-100 proteins |
| 14. | Elevation of IgG, IgM, or IgA antibodies against cross-reactive peptides from dietary |
| | proteins and infectious agents |
| 15. | Elevation of IgG, IgM, or IgA antibodies against glutamate receptor |
| 16. | Elevation of IgG, IgM, or IgA antibodies against phosphodiesterase |

A normal baseline for the tests is obtained by averaging the results for activated T-cells or memory lymphocytes reacting to MS antigens, proinflammatory cytokines, or antibodies against MS antigens for individuals without symptoms relating to multiple sclerosis or other demyelinating diseases. Hence, if an individual exhibits a measurement for activated T-cells or memory lymphocytes reacting to MS antigens, proinflammatory cytokines, or antibodies against MS antigens above the baseline, the above-normal measurement indicates a presence or predisposition to multiple sclerosis and other demyelinating diseases. Preferably, a patient will show above normal measurements for activated T-cells or memory lymphocytes reacting to MS antigens, proinflammatory cytokines, or antibodies against MS antigens; more preferably, a patient will show measurements above about two standard deviations for activated T-cells or memory lymphocytes reacting to MS antigens, proinflammatory cytokines, or antibodies against MS antigens.

Presence or predisposition of multiple sclerosis results in significant levels of activated T-cells or memory lymphocytes reacting to MS antigens, proinflammatory cytokines, or antibodies against MS antigens. The antibodies can be present as IgG, IgM, or IgA.

The test methods of detection of increased amounts of activated T-cells or memory lymphocytes reacting to MS antigens, proinflammatory cytokines, and antibodies against MS antigens can be used to predict a predisposition to multiple sclerosis and other demyelinating diseases. Any test result showing above-normal measurements for activated T-cells or memory lymphocytes reacting to MS antigens, proinflammatory cytokines, or antibodies against MS antigens without symptoms or a clinical diagnosis shows a predisposition to multiple sclerosis or other demyelinating disease.

To test for antibodies to neuronal antigens, an immunoassay can be used. Immunoassays include, but are not limited to, ELISA test, RIA test, latex agglutination, beads assay, and proteomic assays. A preferable immunoassay is the ELISA test. Other immunoassays can be used and the choice of immunoassay can be determined by one of ordinary skill in the art.

To test for amount of lymphokines, a method can be selected from, but not limited to, the following: bioassay, immunoassay, flow cytometry, and RIA. Other methods can be used and the choice of method can be determined by one of ordinary skill in the art.

To test for amount of neuronal antigen- or peptide-specific activated T-cells or neuronal-specific memory lymphocytes, a method can be selected from, but not limited to, the following: flow cytometry and thymidine incorporation. Other methods can be used and the choice of method can be determined by one of ordinary skill in the art.

Furthermore, a combination of clinical test results with the tests for markers, such as activated T-cells or memory lymphocytes reacting to MS antigens, proinflammatory cytokines, and antibodies against MS antigens, can diagnose multiple sclerosis and other demyelinating diseases. Clinical test results can come from MRI, evoked response, and cerebrospinal fluid. For example, a combination of abnormal MRI and evoked response (even with normal cerebrospinal fluid) with activated T-cells or memory lymphocytes reacting to MS antigens and production of proinflammatory cytokines plus antibodies against MS antigens will support the clinical diagnosis of MS in more than 95% of patients, as shown in Table 2. Table 2 shows some possible combinations of test results using clinical data along with testing of markers, such as activated T-cells or memory lymphocytes reacting to MS antigens, proinflammatory cytokines, or antibodies against MS antigens.

TABLE 2
Combination of MRI, Evoked Response with Memory Lymphocytes, Proinflammatory Cytokines
and Neuron-Specific Antibodies for the Diagnosis of Multiple Sclerosis

| | | | | | | |
|---|---|---|---|---|---|---|
| | Evoked | | Memory | Proinflammatory | Specific | |
| MRI | Response | Cerebrospinal | Lymphocytes | Cytokines | Antibodies | Diagnosis |

| | | |
|---|---|---|
| Zero Positive | Not Tested | Normal |
| One or Two Positive | Not Tested | Possible MS |
| All three Positive | Not Tested | Possible MS |
| Zero Positive | Zero Positive | Normal |
| One or Two Positive | Zero Positive | Possible MS |
| All three Positive | Zero Positive | Possible MS |
| Zero Positive | One Positive | Neuroimmune |
| One Positive | One Positive | Possible MS |
| Two Positive | One Positive | MS |
| Three Positive | One Positive | MS |
| Zero Positive | Two Positive | Neuroimmune |
| One Positive | Two Positive | Early MS |
| Two Positive | Two Positive | MS |
| Three Positive | Two Positive | Definite MS |
| Zero Positive | Three Positive | Neuroimmune |
| | | or very early MS |
| One Positive | Three Positive | Early MS |
| Two Positive | Three Positive | Definite MS |
| Three Positive | Three Positive | Definite MS |

The disclosure below is of specific examples setting forth preferred methods for the preferred embodiments. These examples are not intended to limit the scope, but rather to exemplify preferred embodiments.

### EXAMPLE 1

### Materials and Methods

Blood samples from twenty subjects (8 males and 12 females) 32-48 years of age with abnormal MRI and evoked potential and diagnosis of possible MS were sent by different clinicians to our laboratory for neuroimmunological examination. For comparison, blood samples from 40 healthy, age- and sex-matched controls were included in this study.

Myelin basic protein (MBP), myelin associated glycoprotein (MAG), proteolipid protein (PLP), transaldolase, α-β-crystallin, and S-100 proteins were purchased from SIGMA (St. Louis, Mo.). Glial Fibrillary Acidic Protein (GFAP) was purchased from Boeringer Mannheim.

The following peptides were purchased from Research Genetics (Huntsville, Ala.):

Human MBP Peptides:

| | | |
|---|---|---|
| 87-106 | VVHFFKNIVTPRTPPPSQGK | (SEQ ID NO:1) |
| 83-89 | ENPVVHFFKNIVTPRTP | (SEQ ID NO:2) |
| 1-11 | ASQKRPSQRSK | (SEQ ID NO:3) |
| 200-211 | ANMQRQAVPTL | (SEQ ID NO:4) |

Other peptides from 1-250 AA
Proteolipid Protein Peptides

| | | |
|---|---|---|
| 40-60 | TGTEKLIETYFSKNYQDYEYL | (SEQ ID NO:5) |
| 89-106 | GFYTTGAVRQIFGDYKTT | (SEQ ID NO:6) |
| 103-120 | YKTTICGKGLSATVTGGQ | (SEQ ID NO:7) |
| 125-143 | SRGQHQAHSLERVCHCLGK | (SEQ ID NO:8) |
| 139-154 | HCLGKWLGHPDKFVGI | (SEQ ID NO:9) |

Other peptides from 1-250 AA
Transaldolase Peptides

| | | |
|---|---|---|
| 11-25 | MESALDQLKQFTTVV | (SEQ ID NO:10) |
| 21-35 | ETTVVADTGDFHAID | (SEQ ID NO:11) |
| 31-45 | FHAIDEYKPQDATTN | (SEQ ID NO:12) |
| 71-85 | KLGGSQEDQIKNAID | (SEQ ID NO:13) |
| 81-95 | KNAIDKLFVLFGAEI | (SEQ ID NO:14) |
| 261-275 | GELLQDNAKLVPVLS | (SEQ ID NO:15) |
| 271-285 | VPVLSAKAAQASDLE | (SEQ ID NO:16) |
| 311-325 | GIRKFAADAVKLERM | (SEQ ID NO:17) |

Other peptides from 1-337 AA
Myelin Oligodendrocyte Glycoprotein Peptides

| | | |
|---|---|---|
| 1-20 | GQFRVIGPRHPIRALVGDEV | (SEQ ID NO:18) |
| 61-80 | QAPEYRGRTELLKDAIGEGK | (SEQ ID NO:19) |
| 101-120 | RDHSYQEEAAMELKVEDPFY | (SEQ ID NO:20) |
| 145-160 | VFLCLQYRLRGKLRAE | (SEQ ID NO:21) |

Other peptides from 1-218 AA
Myelin Associated Glycoprotein Peptides

| | | |
|---|---|---|
| 37-60 | REIVDRKYSICKSGCFYQKKEEDW | (SEQ ID NO:22) |

Other peptides from 1-81 AA

### EXAMPLE 2

### Enzyme-Linked Immunosorbent Assay (ELISA) Procedure

Enzyme-linked immunosorbent assay (ELISA) was used for testing antibodies against different neuron-specific antigens in the sera of patients with possible MS and control subjects. Antigens or peptides were dissolved in methanol at a concentration of 1.0 mg/ml, then diluted 1:100 in 0.1 M carbonate-bicarbonate buffer, pH 9.5, and 50 μl were added to each well of a polystyrene flat-bottom ELISA plate. Plates were incubated overnight at 4° C. and then washed three times with 20 mm Tris-buffered saline (TBS) containing 0.05% Tween 20, pH 7.4. The nonspecific binding of immunoglobulins was prevented by adding a mixture of 1.5% bovine serum albumin (BSA) and 1.5% gelatin in TBS, and then incubating for 2 h at room temperature, and then overnight at 4° C. Plates were washed as in the above, and then serum samples diluted 1:100 in 1% BSA-TBS were added to duplicate wells and incubated for 2 h at room temperature. Sera from patients with multiple sclerosis, polyneuropathies and other neurological disorders with known high titers of IgG, IgM and IgA against different neurological antigens were used to rule out non-specific antibody activities of inter- and intra-assay variability. Plates were washed, and then peroxidase-conjugated goat anti-human IgG, IgM or IgA antiserum (KPI, Gaithersburg, Md.) diluted 1:400 in 1% BSA-TBS was added to each well; the plate was incubated for an additional 2 h at room temperature. After washing five times with TBS-Tween buffer, the enzyme reaction was started by adding 100 μl of o-phenylene diamine in citrate-phosphate buffer, pH 5.0 and hydrogen peroxide diluted 1:10,000. After 45 min, the reaction was stopped with 50 μl of 2 N H₂SO₄. The optical density (O.D.) was read at 492 nm by means of a microtiter reader. Several control wells containing all reagents, but human serum, were used for detecting nonspecific binding.

### EXAMPLE 3

### Detection of Neurologic Antibodies

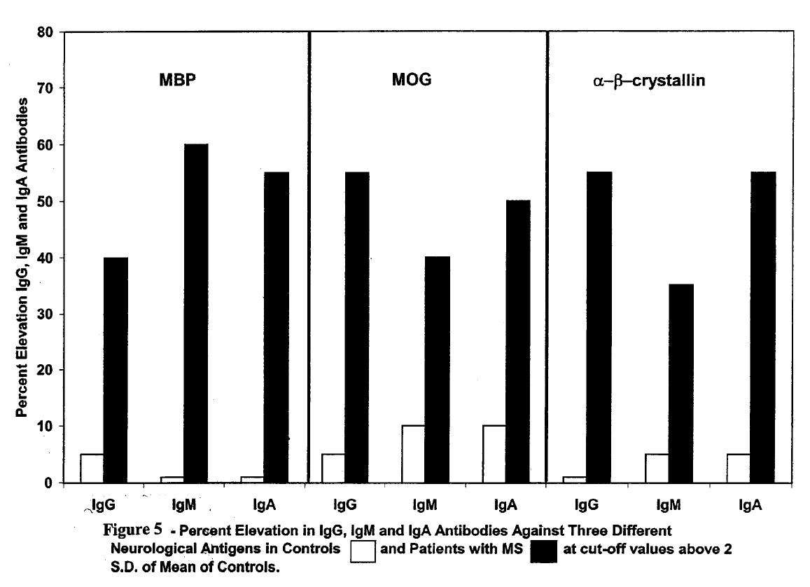

Using ELISA assays, sera from 20 healthy subjects and 20 patients with possible MS were analyzed for the presence of IgG, IgM, and IgA antibodies against three neuron-specific antigens. The ELISA results expressed as mean O.D. at 492 nm are summarized in Table 3. The O.D. for IgG antibody values obtained with 1:100 dilution of healthy control sera ranged from 0.03 to 0.78, varying among subjects and antigens. The mean±standard deviation (S.D.) of these O.D. values, as shown in Table 3, ranged from 0.15±0.06 to 0.19±0.16. The corresponding IgG O.D. values from MS patients sera ranged from 0.06 to 2.27 and with the mean±S.D. of IgG values, which ranged from 0.58±0.49 to 0.75±0.73. For all three antigens, the differences between mean±S.D. of control sera and MS patients sera were highly significant (p<0.001). At a cutoff value of 2 S.D. above the mean of control values, levels of IgG antibody against these antigens were calculated in control and patients sera and found that while 0-5% of control sera had IgG values higher than 2 S.D. of controls, the MS group showed elevated IgG values from 40 to 55% (p<0.001) (FIG. 5).

Levels of IgM antineuron-specific antigens in sera of healthy controls and patients with MS are shown in Table 3. These serum IgM antibodies against all three different tested antigens were significantly higher in patients than in controls. The mean±S.D. for controls ranged from 0.14±0.04 to 0.17±0.10 O.D. and for patients ranged from 0.35±0.29 to 0.47±0.39 O.D. (p<0.001). When the 2 S.D. mean of controls was used as a cut-off point, 0 to 10% of controls versus 35 to 60% of MS patients sera showed elevated IgM antibody levels (p<0.001) (FIG. 5). Likewise, IgA antibody levels against these neurological antigens were examined in both groups. Individual and mean±S.D. data depicted in Table 3 showed significant differences between control and patients groups. The mean±S.D. for IgA antibody levels in controls ranged from 0.12±0.06 to 0.17±0.12 and in patients, from 0.44±0.46 to 0.48±0.42 (p<0.001). Percent elevated serum IgA anti-neuronal autoantibodies at the O.D. value of greater than 2 S.D. of mean controls, was significantly higher in MS patients than in controls. The percent positive for IgA antibodies in controls ranged from 0 to 10% and in patients 50-55% (p<0.001) (FIG. 5).

TABLE 3
Measurement of Antibodies against Neuron-Specific
Antigens in Controls and Patients with Multiple Sclerosis
Expressed by ELISA Optical Densities.

| | Myelin Basic Protein | | | | | |
|---|---|---|---|---|---|---|
| Specimen | IgG | | IgM | | IgA | |
| # | C | P | C | P | C | P |
| 1 | 0.15 | 0.87 | 0.21 | 0.32 | 0.11 | 0.94 |
| 2 | 0.11 | 0.23 | 0.15 | 0.37 | 0.24 | 0.19 |
| 3 | 0.24 | 0.17 | 0.18 | 0.19 | 0.23 | 0.20 |
| 4 | 0.05 | 1.53 | 0.18 | 0.99 | 0.08 | 1.23 |
| 5 | 0.17 | 0.06 | 0.13 | 0.27 | 0.18 | 0.31 |
| 6 | 0.03 | 1.28 | 0.21 | 0.80 | 0.15 | 0.57 |
| 7 | 0.09 | 0.20 | 0.14 | 0.21 | 0.08 | 0.36 |
| 8 | 0.17 | 1.95 | 0.08 | 0.61 | 0.05 | 0.58 |
| 9 | 0.36 | 0.12 | 0.17 | 0.21 | 0.09 | 0.23 |
| 10 | 0.20 | 0.35 | 0.12 | 1.85 | 0.07 | 1.24 |
| 11 | 0.12 | 0.27 | 0.16 | 0.34 | 0.09 | 0.21 |
| 12 | 0.15 | 0.13 | 0.17 | 0.24 | 0.16 | 0.88 |
| 13 | 0.28 | 0.89 | 0.12 | 0.59 | 0.19 | 0.42 |
| 14 | 0.78 | 0.25 | 0.07 | 0.32 | 0.15 | 0.06 |
| 15 | 0.04 | 1.98 | 0.12 | 0.63 | 0.02 | 0.18 |
| 16 | 0.15 | 0.27 | 0.09 | 0.19 | 0.11 | 0.37 |
| 17 | 0.18 | 0.26 | 0.18 | 0.34 | 0.15 | 0.20 |
| 18 | 0.03 | 0.15 | 0.09 | 0.06 | 0.14 | 0.25 |
| 19 | 0.32 | 2.27 | 0.05 | 0.26 | 0.08 | 0.87 |
| 20 | 0.24 | 1.81 | 0.15 | 0.35 | 0.20 | 0.24 |
| Mean ± | 0.19 ± | 0.75 ± | 0.14 ± | 0.46 ± | 0.13 ± | 0.47 ± |
| S.D. | 0.16 | 0.73 | 0.04 | 0.40 | 0.06 | 0.36 |
| | Myelin Oligodendrocytes | | | | | |
| Specimen | IgG | | IgM | | IgA | |
| # | C | P | C | P | C | P |
| 1 | 0.22 | 0.36 | 0.18 | 0.15 | 0.12 | 0.54 |
| 2 | 0.16 | 1.72 | 0.23 | 0.95 | 0.25 | 0.17 |
| 3 | 0.15 | 0.16 | 0.29 | 0.24 | 0.18 | 0.15 |
| 4 | 0.23 | 0.34 | 0.16 | 0.41 | 0.09 | 0.89 |
| 5 | 0.17 | 1.76 | 0.09 | 0.35 | 0.16 | 0.98 |
| 6 | 0.20 | 0.13 | 0.19 | 1.62 | 0.53 | 0.27 |
| 7 | 0.46 | 0.18 | 0.15 | 0.22 | 0.14 | 0.32 |
| 8 | 0.14 | 0.26 | 0.12 | 0.31 | 0.11 | 0.38 |
| 9 | 0.15 | 0.12 | 0.18 | 0.34 | 0.19 | 0.27 |
| 10 | 0.11 | 0.23 | 0.05 | 1.41 | 0.14 | 1.89 |
| 11 | 0.16 | 1.52 | 0.12 | 0.31 | 0.12 | 0.91 |
| 12 | 0.07 | 0.75 | 0.11 | 0.64 | 0.06 | 0.36 |
| 13 | 0.05 | 0.92 | 0.18 | 0.61 | 0.21 | 0.83 |
| 14 | 0.13 | 0.22 | 0.15 | 0.19 | 0.44 | 0.13 |
| 15 | 0.28 | 1.34 | 0.49 | 0.21 | 0.15 | 0.36 |
| 16 | 0.03 | 0.22 | 0.24 | 0.17 | 0.13 | 0.41 |
| 17 | 0.07 | 0.09 | 0.13 | 0.28 | 0.08 | 0.27 |
| 18 | 0.16 | 0.35 | 0.14 | 0.33 | 0.05 | 0.15 |
| 19 | 0.05 | 1.61 | 0.35 | 0.45 | 0.09 | 0.25 |
| 20 | 0.09 | 0.98 | 0.02 | 0.24 | 0.14 | 0.20 |
| Mean ± | 0.15 ± | 0.66 ± | 0.17 ± | 0.47 ± | 0.17 ± | 0.48 ± |
| S.D. | 0.09 | 0.60 | 0.10 | 0.39 | 0.12 | 0.42 |

| | α-β-Crystallin | | | | | |
|---|---|---|---|---|---|---|
| Specimen | IgG | | IgM | | IgA | |
| # | C | P | C | P | C | P |
| 1 | 0.26 | 0.29 | 0.17 | 0.38 | 0.14 | 0.53 |
| 2 | 0.18 | 0.12 | 0.26 | 0.22 | 0.31 | 0.22 |
| 3 | 0.14 | 0.23 | 0.13 | 0.07 | 0.12 | 0.15 |
| 4 | 0.10 | 1.35 | 0.04 | 0.13 | 0.17 | 0.87 |
| 5 | 0.11 | 0.38 | 0.15 | 0.26 | 0.19 | 0.32 |
| 6 | 0.13 | 0.24 | 0.11 | 0.32 | 0.17 | 0.09 |
| 7 | 0.18 | 0.51 | 0.19 | 0.98 | 0.02 | 1.31 |
| 8 | 0.21 | 0.27 | 0.56 | 0.20 | 0.14 | 0.11 |
| 9 | 0.12 | 0.29 | 0.08 | 0.17 | 0.04 | 0.36 |
| 10 | 0.09 | 1.15 | 0.18 | 0.24 | 0.06 | 0.15 |
| 11 | 0.05 | 0.36 | 0.21 | 0.35 | 0.15 | 0.86 |
| 12 | 0.14 | 0.28 | 0.08 | 0.24 | 0.13 | 0.18 |
| 13 | 0.23 | 1.34 | 0.16 | 0.69 | 0.11 | 0.27 |
| 14 | 0.18 | 0.27 | 0.12 | 0.14 | 0.08 | 0.22 |
| 15 | 0.11 | 0.98 | 0.18 | 0.26 | 0.09 | 0.33 |
| 16 | 0.08 | 0.36 | 0.16 | 0.25 | 0.17 | 0.45 |
| 17 | 0.29 | 1.87 | 0.14 | 1.24 | 0.02 | 1.94 |
| 18 | 0.11 | 0.09 | 0.12 | 0.34 | 0.07 | 0.24 |
| 19 | 0.18 | 0.89 | 0.03 | 0.33 | 0.14 | 0.28 |
| 20 | 0.10 | 0.47 | 0.24 | 0.26 | 0.11 | 0.09 |
| Mean ± | 0.15 ± | 0.58 ± | 0.16 | 0.35 ± | 0.12 ± | 0.44 ± |
| S.D. | 0.06 | 0.49 | 0.11 | 0.29 | 0.06 | 0.46 |

### EXAMPLE 4

### Assay Variation of IgG, IgM, IgA

Coefficients of interassay variation were calculated by running five samples eight times in one assay. Coefficients of interassay variation were determined by measuring the same samples in six consecutive assays. This replicate testing established the validity of the ELISA assays, determined the appropriate dilution with minimal background and detected serum IgG, IgM and IgA against different antigens. Two sera from healthy controls, two nonspecific sera from MS patients and two sera from autistic children were used to construct standard control curves. These sera were diluted 1:25, 1:50, 1:100, 1:200 and 1:400. At dilutions of 1:50-1:200, the standard curve for MS sera was linear and antibodies from healthy controls were not detected against the three tested antigens. Coefficients of intra-assay variations for IgG, IgM, and IgA against the three antigens were less than 8%. Coefficients of interassay variations were less than 10%.

### EXAMPLE 5

### Lymphocyte Proliferation Assay and Cytokine Production

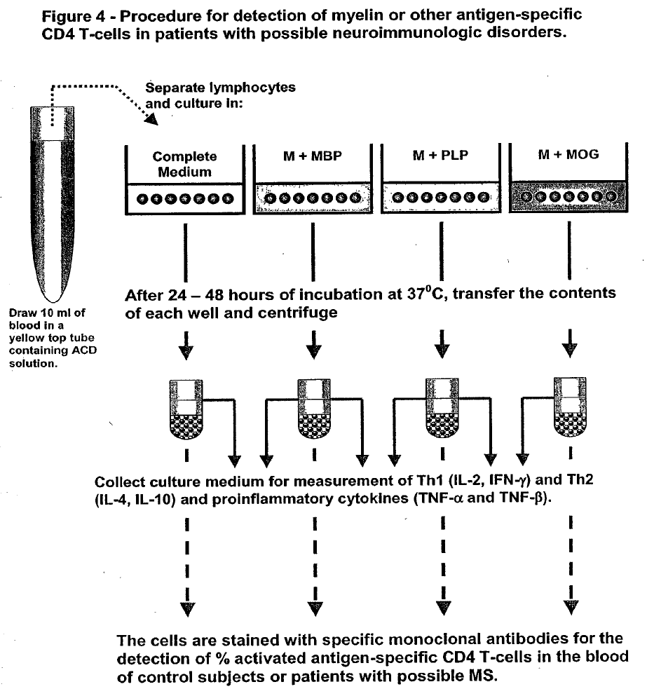

Peripheral blood mononuclear cells (PBMCs) were isolated from blood drawn in ACD yellow top tubes by Ficoll Density Centrifugation (SIGMA, St. Louis, Mo.). PBMCs were incubated at a cell density of 1×10⁶/ml in complete RPMI alone or in complete RPMI (CRPMI) containing different neuronal antigens or peptides, at a final concentration of 10 μg/ml. After 48 hours incubation at 37° C., the contents of each well was transferred to a separate tube and centrifuged at 1,500 g. The cells were labeled with CD25+CD69 monoclonal antibodies and % antigen-specific CD3 activated T-cells were measured by flow cytometry (Becton Dickinson FacScan). The stimulation index was calculated by dividing the reactive well containing cells+antigen by controls containing only cells in complete medium. Supernatant was removed and used for measurement of TH₁ (IL-2, IFN-γ), TH₂ (IL-4, IL-10) and proinflammatory cytokines (TNF-α and TNF-β). Cytokine concentrations were measured in picograms per ml of cell culture supernatants by ELISA, using kits manufactured by Biosourse International (Camarillo, Calif.). A summary of this procedure for the measurement of neuronal antigen-activated lymphocyte and cytokine production is shown in FIG. 4.

### EXAMPLE 6

### Detection of Neurological Antigen-Specific Reactive T-Cells

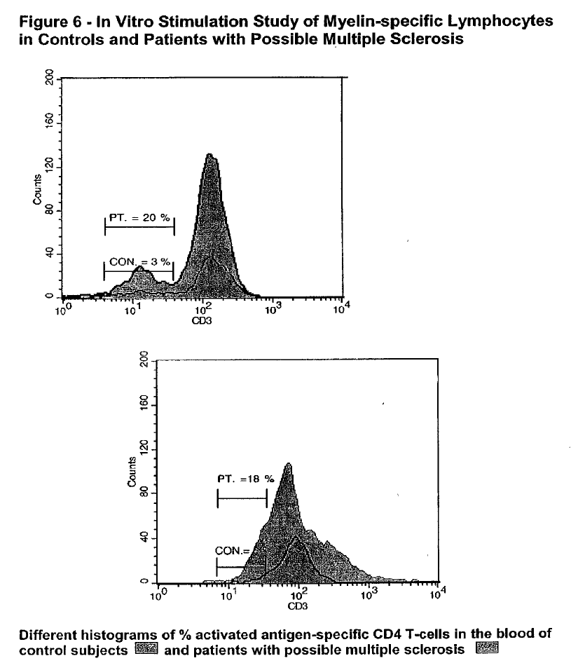

MBP, MOG and α-β-crystallin reactive T-cells were tested in a proliferation assay. Histogram of two controls with 3% and 6% and two patients with 20% and 18% of MBP-reactive T-cells are shown in FIG. 6. The percentage of reactive T-cells of controls and patients cultured in medium alone or medium+MBP, medium+MOG and medium+α-β-crystallin are shown in Table 4. Comparison of individual values of controls and patients with MS, showed significant differences in their lymphocyte reactivity without antigenic stimulation. The mean±S.D. of this spontaneous T-cell reactivity in controls was 4.2±2.2 and for patients, 8.6±3.4 (p<0.05).

The percentage of MBP, reactive T-cells of controls ranged from 1-12% with mean±S.D. of 5.0±2.4; MOG was 2-9% with mean±S.D. of 4.9±2.1; and α-β-crystallin was 1-8% at 4.2±1.8. The corresponding values in MS patients ranged from 4-35% with mean±S.D. of 18.4±9.8 for MBP; MOG was 6-27% with mean±S.D. of 15.1±6.4; and α-β-crystallin was 5-21% at 10.7±4.5. The differences between lymphocyte reactivity to all tested neurological antigens in controls and MS patients were highly significant (P<0.001). The pattern of lymphocyte reactivity varied from antigen to antigen in different patients (Table 4). Some reacted to none of the antigens, ore reacted only to MBP or to a combination of MBP+MOG, MBP+α-β-crystallin or to MBP+MOG+α-β-crystallin.

TABLE 4
Percent Memory Lymphocyte Immune Stimulation Assay in Medium
Alone (M) or M + MBP, M + MOG and M + α-β-crystallin
in Controls (C) and Patients (P) with Multiple Sclerosis,
Performed by Culture and Flow Cytometry

| Specimen | Medium (M) | | M + MBP | | M + MOG | | M + α-β-crystallin | |
|---|---|---|---|---|---|---|---|---|
| # | C | P | C | P | C | P | C | P |
| 1 | 2.0 | 9.0 | 3.0 | 20.0 | 5.0 | 14.0 | 6.0 | 18 |
| 2 | 5.0 | 11.0 | 6.0 | 18.0 | 4.0 | 17.0 | 2.0 | 9.0 |
| 3 | 3.0 | 12.0 | 4.0 | 27.0 | 6.0 | 21.0 | 5.0 | 15.0 |
| 4 | 5.0 | 13.0 | 7.0 | 25.0 | 2.0 | 16.0 | 4.0 | 11.0 |
| 5 | 2.0 | 5.0 | 4.0 | 8.0 | 3.0 | 6.0 | 2.0 | 5.0 |
| 6 | 1.0 | 6.0 | 3.0 | 7.0 | 4.0 | 8.0 | 1.0 | 7.0 |
| 7 | 3.0 | 15.0 | 5.0 | 35.0 | 2.0 | 27.0 | 6.0 | 14.0 |
| 8 | 6.0 | 10.0 | 12.0 | 28.0 | 8.0 | 14.0 | 4.0 | 12.0 |
| 9 | 4.0 | 5.0 | 8.0 | 6.0 | 7.0 | 9.0 | 8.0 | 10.0 |
| 10 | 7.0 | 6.0 | 5.0 | 15.0 | 9.0 | 18.0 | 6.0 | 11.0 |
| 11 | 1.0 | 8.0 | 4.0 | 21.0 | 3.0 | 17.0 | 5.0 | 16.0 |
| 12 | 3.0 | 4.0 | 2.0 | 13.0 | 5.0 | 15.0 | 4.0 | 12.0 |
| 13 | 5.0 | 12.0 | 6.0 | 29.0 | 8.0 | 23.0 | 2.0 | 14.0 |
| 14 | 2.0 | 7.0 | 5.0 | 17.0 | 6.0 | 11.0 | 3.0 | 8.0 |
| 15 | 6.0 | 9.0 | 3.0 | 16.0 | 4.0 | 10.0 | 5.0 | 6.0 |
| 16 | 5.0 | 4.0 | 4.0 | 10.0 | 5.0 | 9.0 | 3.0 | 7.0 |
| 17 | 3.0 | 11.0 | 1.0 | 33.0 | 2.0 | 24.0 | 4.0 | 21.0 |
| 18 | 4.0 | 5.0 | 6.0 | 5.0 | 7.0 | 9.0 | 5.0 | 8.0 |
| 19 | 9.0 | 14.0 | 8.0 | 30.0 | 6.0 | 26.0 | 7.0 | 6.0 |
| 20 | 8.0 | 6.0 | 5.0 | 4.0 | 3.0 | 8.0 | 2.0 | 5.0 |
| Mean ± | 4.2 ± | 8.6 ± | 5.0 ± | 18.4 ± | 4.9 ± | 15.1 ± | 4.2 ± | 10.7 ± |
| S.D. | 2.2 | 3.4 | 2.4 | 9.8 | 2.1 | 6.4 | 1.8 | 4.5 |

C = control
P = patient

### EXAMPLE 7

### Cytokine Production

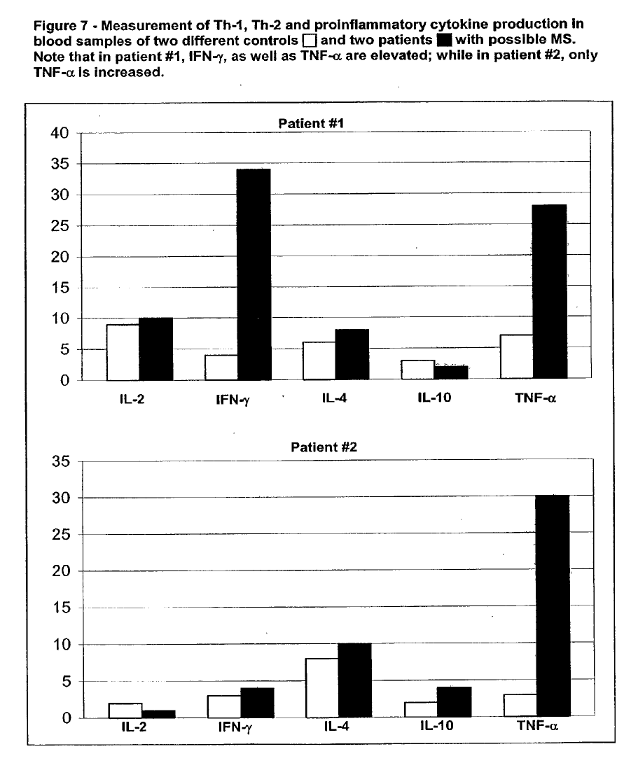

Cytokine production of cell culture supernatants from MBP-reactive T-cells were determined by ELISA and expressed by picograms/ml. This pattern of cytokine production in supernatants of two controls and two MS patients is illustrated in FIG. 6 and 20 controls and 20 MS patients in Table 5. As shown in FIG. 7, patient 1 produced significant levels of TNF-α and IFN-γ while patient 2 produced high levels of TNF-α but not IFN-γ. Furthermore, analysis of cytokine levels in all 20 controls and patients, showed TNF-α first, with mean±S.D. of 24.7±15.0, then IFN-γ with mean±S.D. of 20±16.6 and TNF-β levels with mean±S.D. of 13.8±10.4. Production of these cytokines by activated T-cells was significantly above the background levels produced by controls lymphocytes (p<0.001). For IL-2, IL-4 and IL-10, the differences between controls and patients were not significant (Table 5). The percent of elevated cytokine production by different MS patients and controls at 2 S.D. above the mean values of controls, were analyzed and found to be significantly higher in MS patients than in controls. The percent of elevation for TNF-β, TNF-α, and IFN-γ production in controls ranged from 5-10%, and in patients was at 40%, 70% and 75%, respectively (FIG. 7).

TABLE 5
Measurement of T-helper-1/T-helper-2 and Proinflammatory
Cytokines after 48 Hours Culture of Human Lymphocytes
with Myelin-Basic Protein, Myelin-Oligodendrocytes and
α-β-Crystallin Expressed by picogram/ml.

| Specimen | Interleukin-2 | | Interferon-γ | | Interleuken-4 | |
|---|---|---|---|---|---|---|
| # | C | P | C | P | C | P |
| 1 | 9.0 | 10.0 | 4.0 | 34.0 | 6.0 | 8.0 |
| 2 | 2.0 | 1.0 | 1.0 | 4.0 | 3.0 | 10.0 |
| 3 | 5.0 | 11.0 | 4.0 | 8.0 | 4.0 | 7.0 |
| 4 | 3.0 | 6.0 | 3.0 | 28.0 | 8.0 | 10.0 |
| 5 | 10.0 | 16.0 | 3.0 | 51.0 | 3.0 | 7.0 |
| 6 | 1.0 | 9.0 | 7.0 | 11.0 | 6.0 | 4.0 |
| 7 | 6.0 | 8.0 | 2.0 | 27.0 | 9.0 | 14.0 |
| 8 | 5.0 | 2.0 | 1.0 | 9.0 | 5.0 | 3.0 |
| 9 | 7.0 | 5.0 | 2.0 | 36.0 | 8.0 | 6.0 |
| 10 | 3.0 | 1.0 | 3.0 | 12.0 | 4.0 | 9.0 |
| 11 | 6.0 | 8.0 | 10.0 | 9.0 | 3.0 | 7.0 |
| 12 | 2.0 | 5.0 | 8.0 | 19.0 | 5.0 | 4.0 |
| 13 | 3.0 | 7.0 | 3.0 | 6.0 | 3.0 | 2.0 |
| 14 | 8.0 | 10.0 | 1.0 | 44.0 | 9.0 | 12.0 |
| 15 | 5.0 | 9.0 | 6.0 | 2.0 | 3.0 | 15.0 |
| 16 | 1.0 | 14.0 | 3.0 | 4.0 | 2.0 | 7.0 |
| 17 | 12.0 | 18.0 | 7.0 | 31.0 | 6.0 | 4.0 |
| 18 | 2.0 | 8.0 | 2.0 | 16.0 | 5.0 | 9.0 |
| 19 | 7.0 | 6.0 | 3.0 | 6.0 | 7.0 | 13.0 |
| 20 | 4.0 | 15.0 | 11.0 | 58.0 | 8.0 | 10.0 |
| Mean ± | 5.0 ± | 8.4 ± | 4.0 ± | 20.7 ± | 5.6 ± | 8.0 ± |
| S.D. | 2.8 | 4.2 | 3.0 | 16.6 | 2.2 | 3.8 |

| Specimen | Interleukin-10 | | TNF-α | | TNF-β | |
|---|---|---|---|---|---|---|
| # | C | P | C | P | C | P |
| 1 | 3.0 | 2.0 | 7.0 | 28.0 | 3.0 | 8.0 |
| 2 | 2.0 | 4.0 | 3.0 | 30.0 | 7.0 | 5.0 |
| 3 | 1.0 | 1.0 | 3.0 | 3.9 | 5.0 | 18.0 |
| 4 | 2.0 | 4.0 | 3.0 | 26.0 | 9.0 | 41.0 |
| 5 | 3.0 | 1.0 | 9.0 | 7.0 | 8.0 | 23.0 |
| 6 | 5.0 | 7.0 | 6.0 | 26.0 | 4.0 | 17.0 |
| 7 | 4.0 | 2.0 | 12.0 | 41.0 | 2.0 | 14.0 |
| 8 | 5.0 | 1.0 | 4.0 | 16.0 | 6.0 | 12.0 |
| 9 | 3.0 | 6.0 | 3.0 | 47.0 | 16.0 | 27.0 |
| 10 | 8.0 | 4.0 | 5.0 | 33.0 | 8.0 | 15.0 |
| 11 | 7.0 | 16.0 | 2.0 | 7.0 | 7.0 | 3.0 |
| 12 | 6.0 | 4.0 | 8.0 | 22.0 | 5.0 | 14.0 |
| 13 | 10.0 | 8.0 | 3.0 | 6.0 | 4.0 | 29.0 |
| 14 | 3.0 | 4.0 | 9.0 | 38.0 | 11.0 | 5.0 |
| 15 | 2.0 | 5.0 | 4.0 | 29.0 | 6.0 | 3.0 |
| 16 | 1.0 | 3.0 | 1.0 | 12.0 | 7.0 | 10.0 |
| 17 | 6.0 | 4.0 | 16.0 | 33.0 | 5.0 | 21.0 |
| 18 | 2.0 | 13.0 | 4.0 | 15.0 | 14.0 | 6.0 |
| 19 | 4.0 | 9.0 | 2.0 | 5.0 | 8.0 | 3.0 |
| 20 | 6.0 | 5.8 | 7.0 | 38.0 | 7.0 | 2.0 |
| Mean ± | 4.1 ± | 5.2 ± | 5.5 ± | 24.7 ± | 7.1 ± | 13.8 ± |
| S.D. | 2.4 | 3.9 | 3.7 | 15.0 | 3.4 | 10.4 |

C = control
P = patient

In this analysis, IFN-γ, TNF-α, and TNF-β were considered to be produced by TH₁ cells, IL-4 by TH₂ cells, and IL-10 by both subsets, except at lower levels in which case they are produced by TH₁ cells. TH₀ cells produce both IL-4 and IFN-γ. Compared with unaffected individuals, the MBP-reactive T-cells in MS patients exhibited TH₁ cytokine profiles (Table 5 and FIG. 7).

Many modifications and variations of the embodiments described herein may be made without departing from the scope, as is apparent to those skilled in the art. The specific embodiments described herein are offered by way of example only.

### REFERENCES

1. Noronha, A., Arnanson, B. Demyelinating diseases. In Clinical Immunology. (Rich, Fleisher, Schwartz, Shearer and Strober Eds.) Mosby, pp. 1364-1375, 1995.
2. Steinman, L. Multiple sclerosis: a coordinated immunological attack against myelin in the central nervous system. Cell 85:299, 1996.
3. Amor, S., Baker, D., Layward, L., McCormack, K., Van Noort, J. M. Multiple Sclerosis: Variations on a Theme. Immunology Today 18:368-371, 1997.
4. Raine, C. S., Canella, B., Hauser, S. L., Genain, C. P. Demyelination in primate autoimmune encephalomyelitis and acute multiple sclerosis lesions: a case for antigen-specific antibody mediation. Ann. Neurol. 46:144, 1999.
5. Noseworthy, J. H., Lucchinetti, C., Rodriguez, M., Weinshenker, B. G. Multiple Sclerosis, a review. New England Journal of Medicine 343 :938-952, 2000.
6. Moktarian, F., Zhang, Z., Shi, Y., Gonzalez, E., Sobel, R. A. Molecular mimicry between a viral peptide and a myelin oligodendrocyte glycoprotein peptide induces autoimmune demyelinating disease in mice. J. Neuroimmunol. 95:43, 1999.
7. Esposito M., Venkatesh, V., Otvos, L., Weng, Z., Vajda, S., Banki, K., Perl, A. Human transaldolase and cross-reactive viral epitopes identified by autoantibodies of multiple sclerosis patients. J. of Immunology 163:4027-4032, 1999.
8. Genain, C. P., Cannella, B., Hauser, S. L., Raine, C. S. Identification of autoantibodies associated with myelin damage in multiple sclerosis. Nature Med. 5: 170-175, 1999.
9. Holz, A., Bielekova, B., Martin, R., Oldstone M. B. A. Myelin-associated oligodendrocytic basic protein: Identification of an encephalitogenic epitope and association with multiple sclerosis. J. Immun. 164:1103-1109, 2000.
10. Stefferl, A., Schubart, A., Storch, M., Amini, A. Mather, I., Lassmann, H., Linington, C. Butrophilin, a milk protein, modulates the encephalitogenic T-cell response to myelin oligodendrocyte glycoprotein in experimental autoimmune encephalomyelitis. J. Immunol. 165:2859-2865, 2000.
11. Bajramovic, J. J. et al. Presentation of α-β-crystallin to T-cells in active multiple sclerosis lesions: an early event following inflammatory demyelination. J. of Immunology 164:4359-4366, 2000.
12. Brock, H. P. M., Uccelli, A., Kerlero de Rosbo, N., Bontrop, R. E., Roccatagliata, L., DeGroot, N. G., Capello, E., Laman, J. D., Nicolay, K., Mancardi, G., Ben-Nun, A., 't Hart, B. A. Myelin/oligodendrocyte glycoprotein-induced autoimmune encephalomyelitis in common marmosets: the encephalitogenic T-cell epitope P-MOG 24-36 is presented by a monomorphic MHC class II molecule. Journal of Immunology 165:1093-1101, 2000.
13. Von Büdingen, H., Tanuman, N., Villaslada, P., Ouallet, J., Hauser, S. L., Genain, C. P. Immune response against the myelin/oligodendrocyte glycoprotein in experimental autoimmune demyelination. J. Clin. Immunology 21:155, 2001.
14. Chou, Y. K., Bourdette, D. N., Offner, H., Witham, R., Wang, Y., Hashim, G. A., Vandenbark, A. A. Frequency of T-cells specific for myelin basic protein and myelin proteolipid protein in blood and cerebrospinal fluid in multiple sclerosis. J. Neuroimmunol. 38:105, 1992.
15. Kerlero de Rosbo, N. R., Milo, R., Lees, M. B., Burger, D., Bernard, C. C., Ben-Nun, A. Reactivity to myelin antigens in multiple sclerosis: peripheral blood lymphocytes respond predominantly to myelin oligodendrocyte glycoprotein. J. Clin. Invest. 92:2602, 1993.
16. Genain, C. P., Lee-Parritz, D., Nguyen, M. H., Massacesi, L., Joshi, N., Ferrante, R., Hoffman, K., Moseley, M., Letvin, N. L., Hauser, S. L. In healthy primates, circulating autoreactive T-cells mediate autoimmune disease. J. Clin. Invest. 94: 1339, 1994.
17. Correale, J. M., McMillan, M., McCarthy, K., Le, T. Weiner, L. P. Isolation and characterization of autoreactive proteolipid protein-peptide specific T-cell clones from multiple sclerosis patients. Neurology 45:1370.
18. 't Hart, B. A., Van Meurs, M., Brock, H. P. M., Massaces, L., Bauer, J., Boon, L., Bontrop, R. E., Laman, J. D. A New Primate Model for Multiple Sclerosis in the Common Marmoset. Immunology Today 21:290, 2000.
19. Sadatipour, B. T., Greer, J. M., Pender, M. P. Increased circulating antiganglioside antibodies in primary and secondary progressive multiple sclerosis. Ann. Neurol. 44:980, 1998.
20. Grogan, J. L., Kramer, A., Nogai, A., Dong, L., Ohde, M., Schneider-Mergener, J., Kamrad, T. T. Cross-reactivity of myelin basic protein-specific T-cells with multiple microbial peptides: Experimental autoimmune encephalomyelitis induction in TCR transgenic mice. J. Immunol 163:3764-3770, 1999.
21. Xiao, B., Link, H. Is there a balance between microglia and astrocytes in regulating Th1/Th2-cell responses and neuropathologies? Immunology Today 20:477-479, 1999.
22. Singh, R. K., Zang, Y. C. Q., Shrivasta, A., Hong, J., Wang, G. T., Li, S., Tejada-Simon, M. V., Kozovska, M., Rivera, V. M., Zhang, J. Z. Th-1 and Th-2 deviation of myelin-autoreactive T-cells by altered peptide ligands is associated with reciprocal regulation of Lck, Fyn, and ZAP-70. J. Immunol. 163:6393-6402, 1999.
23. Hellings, N., Baree, M., Verhoeven, C., D'hooghe, B., Medaer, R., Bernard, C. C., Raus, J., Stinissen, P. T-cells reactivity to multiple myelin antigens in multiple sclerosis patients and healthy controls. J. Neurosci. Res. 63:290, 2001.
24. Yura, M., Takahashi, I., Serada, M., Koshio, T., Nakagami, K., Yuki, Y., Kiyono, H. Role of MOG-stimulated Th-1 type ‘lightup’ (GFP⁺) CD4⁺ T-cells for the development of experimental autoimmune encephalomyelitis (EAE). J. Autoimmunity 17:17-25, 2001.
25. Kuchroo, V. K., Anderson, A. C., Waldner, H., Munder, M., Bettelli, E., Nicholson, L. B. T-cell response in experimental autoimmune encephalomyelitis (EAE); role of self and cross-reactive antigens in shaping, tuning, and regulating the autopathogenic T-cell repertoire. Annu. Rev. Immunol. 20:101, 2002.
26. Koehler, N. K. U., Genain, C. P., Giesser, B., Hauser, S. L. The human T-cell response to myelin oligodendrocyte glycoprotein: a multiple sclerosis family-based study. J. Immunol. 168:5920-5927, 2002.
27. Beck, J. Rondot, P., Catinot, L., Falcoff, E., Kirchner, H., Wietzerbin, J. Increased production of interferon-γ and tumor necrosis factor precedes clinical manifestation in multiple sclerosis: do cytokines trigger off exacerbations? Acta Neurol. Scan. 78:318, 1988.
28. Powell, M. B., Mitchell, D., Lederman, J., Buckmeier, J. Zamvil, S. S., Graham, M., Ruddle, N. H., Steinman, L. Lymphotoxin and tumor necrosis factor-α production by myelin basic protein-specific T-cell clones correlates with encephalitogenicity. Int. Immunol. 2:539, 1990.
29. Benvenuto, R., Paroli, M., Buttinelli, C., Franco, A., Barnaba, V., Fieschi, C., Balsano, F. Tumor necrosis factor-α and interferon-γ synthesis by cerebrospinal fluid-derived T-cell clones in multiple sclerosis. Ann. NY Acad. Sci. 650:341, 1992.
30. Correale, J., Gilmore, W., McMillan, M., Li, S., McCarthy, K., Le, T., Weiner, L. P. Patterns of cytokine secretions by autoreactive proteolipid protein-specific T-cell clones during the course of multiple sclerosis. J. Immunol. 154:2959, 1995.
31. Navikas, V., He, B., Link, J., Haglund, M., Soderstrom, M., Fredrikson, S., Ljungdahl, A., Hojeberg, J., Qiao, J., Olsson, T., Link., H. Augmented expression of tumour necrosis factor-α and lymphotoxin in mononuclear cells in multiple sclerosis and optic neuritis. Brain 119:213, 1996.
32. Hermans., G., P., Stinissen, G. P., Hauben, L., Van den Berg-Loonen, E., Raus, J., Zhang, J. Cytokine profile of myelin basic protein-reactive T-cells in multiple sclerosis and healthy individuals. Ann. Neurol. 42:18, 1997.
33. van Oosten, B. W., Brakhof, F., Scholten, P. E., von Blomberg, B. M., Ader, H. J., Polman, C. H. Increased production of tumor necrosis factor-α, and not of interferon-γ, preceding disease activity in patients with multiple sclerosis. Arch. Neurol. 55:793, 1998.
34. Tejada-Simon, M. V., Hong, J., Rivera, V. M., Zhang, J. Z. Reactivity pattern and cytokine profile of T-cells primed by myelin peptides in multiple sclerosis and healthy individuals. Eur. J. Immunol. 31:907, 2001.
35. Wildbaum, G., Netzer, N., Karin, N. Plasmid encoding IFN-γ-inducible protein-10 redirects antigen-specific T-cell polarization and suppresses experimental autoimmune encephalomyelitis. J. Immunol. 168:5885-5892, 2002.
36. Wucherpfenning, K. W. Infectious triggers for inflammatory neurological diseases. Nature Medicine 8:455-457, 2002.
37. Levin, M. C., Lee, S. M., Klaume, F., Morcos, Y., Dohan, F. C., Hasty, K. A., Callaway, J. C., Zunt, J., Desiderio, D. M., Stuart, J. M. Autoimmunity due to molecular mimicry as a cause of neurological disease. Nature Medicine 8:509-513, 2002.
38. Miller, S. D., Olson J. K., Croxford, J. L. Multiple pathways to induction of virus-induced autoimmune demyelination: lessons from Theiler's virus infection. J. Autoimmunity 16:219-227, 2001.
39. Martin, R., Gran, B., Zhao, Y., Markovic-Plese, S., Bielekova, B., Marques, A., Sung, M., Hemmer, B., Simon, R., McFarland, H. F., Pinilla, C. Molecular mimicry and antigen-specific T-cell responses in multiple sclerosis and chronic CNS Lyme disease. J. Autoimmunity 16:187-192, 2001.
40. Vojdani, A., Campbell, A. W., Anyanwu, E., Kashanian, A., Bock, K., Vojdani, E. Antibodies to neuron-specific antigens in children with autism: possible cross-reaction with encephalitogenic proteins from milk, Chlamydia pneumoniae and Streptococcus group A. J. Neuroimmunol, 2002 (in press).

What is claimed is:

1. A method for diagnosing the likelihood and severity of multiple sclerosis in a patient, comprising the steps of:
a) determining a level of antibodies against α-B-crystallin in a sample from the patient;
b) comparing the level of antibodies determined in step a) with a normal level of the antibodies in control patients;
c) determining an amount of neuronal α-B-crystallin-specific activated T-cells or neuronal-specific memory lymphocytes in a sample from the patient;
d) obtaining a stimulation index from step c); and
e) comparing the stimulation index from step d) with a normal stimulation index, wherein:
(i) a normal level or lower than normal level of both stimulation index and antibodies for said α-B-crystallin indicate control patient conditions;
(ii) higher than normal levels of either stimulation index or antibodies for said α-B-crystallin indicate a possibility of multiple sclerosis; and
(iii) higher than normal levels of both stimulation index and antibodies for said α-B-crystallin indicate a likelihood of multiple sclerosis.

2. The method according to claim 1, wherein the normal level of antibodies is calculated by taking a mean of levels of antibodies in individuals without symptoms relating to multiple sclerosis.

3. The method according to claim 1, wherein the higher than normal level of antibodies is higher than about two standard deviations of normal level of antibodies of a control group.

4. The method according to claim 1, wherein determining the level of antibodies in any or all of steps a) and b) is accomplished using an immunoassay.

5. The method according to claim 4, wherein the immunoassay is an enzyme-linked immunosorbent assay.

6. The method according to claim 1, wherein the antibodies are selected from the group consisting of IgG, IgA, and IgM.

7. The method according to claim 1, wherein the normal stimulation index is calculated by taking a mean of stimulation indices in individuals without symptoms relating to multiple sclerosis.

8. The method according to claim 1, wherein the higher than normal stimulation index is higher than about two standard deviations of normal stimulation index of a control group.

9. The method according to claim 1, wherein the T-cells are antigen-specific CD3 activated T-cells.

10. The method of claim 1, further comprising a step of obtaining a clinical test result selected from the group consisting of MRI, evoked response, and cerebrospinal fluid.

11. The method of claim 1, further comprising a step of obtaining at least two clinical test results selected from the group consisting of MRI, evoked response, and cerebrospinal fluid.

12. The method of claim 1, further comprising a step of obtaining clinical test results of MRI, evoked response, and cerebrospinal fluid.

13. The method of claim 1, wherein the diagnosis is made according to Table 2.
